(19) United States
(12) Patent Application Publication
(10) Pub. No.: US 2018/0058996 A1
(43) Pub. Date: Mar. 1, 2018

(54) AIR QUALITY MONITORING SYSTEM FOR A MOTOR VECHICLE

(71) Applicant: FORD GLOBAL TECHNOLOGIES, LLC, Dearborn, MI (US)

(72) Inventors: William Stewart Johnston, South Lyon, MI (US); Lawrence C. Karas, New Boston, MI (US); Upendra J. Patel, Canton, MI (US)

(21) Appl. No.: 15/253,099

(22) Filed: Aug. 31, 2016

(51) Int. Cl. G01N 15/06; G01N 33/00; G01N 1/22

(52) U.S. Cl. CPC G01N 15/06; G01N 2015/0046; G01N 1/2205; G01N 33/0004

(57) ABSTRACT

An air quality monitoring system for a motor vehicle includes a plurality of air sampling points on the motor vehicle, a single air quality sensor and a conduit system connecting the plurality of air sampling points with the single air quality sensor.

### TECHNICAL FIELD

This document relates generally to the motor vehicle equipment field and, more particularly, to an air quality monitoring system for a motor vehicle providing a plurality of air sampling points and a single air quality sensor for testing the air quality at those sampling points.

### BACKGROUND

Air quality is becoming a bigger and bigger concern to individuals seeking to maintain a healthy lifestyle. This includes drivers of motor vehicles. In order to address this issue, motor vehicle manufacturers are investigating methods to measure particulate matter both within the passenger compartment of a motor vehicle and in the ambient environment outside of the passenger compartment of the motor vehicle.

This document relates to a new and improved air quality monitoring system that functions to monitor air quality at a plurality of air sampling points on a motor vehicle utilizing a single air quality sensor. Such an air quality sensor may take the form of a particulate sensor capable of measuring particulates of 2.5 μm and below which have been proven to be hazardous to human health as such particulates are able to penetrate deep within the lungs and cause health issues. As further disclosed, the air quality monitoring system may be utilized in conjunction with the heating, ventilating and air conditioning (HVAC) system of the motor vehicle to both monitor and control air quality in the passenger compartment of the motor vehicle for the benefit of the motor vehicle occupants.

### SUMMARY

In accordance with the purposes and benefits described herein, an air quality monitoring system is provided for a motor vehicle. That air quality monitoring system comprises a plurality of air sampling points on the motor vehicle, a single air quality sensor and a conduit system connecting the plurality of air sampling points with the single air quality sensor. The air quality monitoring system may also include a conduit system having a switching mechanism and a plurality of hoses that extend from the switching mechanism to the plurality of air sampling points.

The air quality monitoring system may also include an air pump. The single air quality sensor and the air pump may be provided downstream from the switching mechanism. Further, an air sample hose may be provided to connect the switching mechanism with the single air quality sensor and the air pump. This allows for the testing of the air quality of a discrete air sample taken from any one of the air sampling points using the single air quality sensor.

The air quality monitoring system may further include a controller configured to control the switching mechanism and allow the discrete air sample to be drawn from any one of the sampling points by the air pump. That controller may be further configured to control the air pump.

In some embodiments, at least one of the plurality of sampling points is in a passenger compartment of the motor vehicle and at least one of the plurality of sampling points is exterior to the passenger compartment on the motor vehicle.

In one particularly useful embodiment of the air quality monitoring system, a first air sampling point is provided in a passenger compartment of the motor vehicle. The air quality monitoring system may also include a second air sampling point in the passenger compartment of the motor vehicle. In such an embodiment the first air sampling point may be provided motor vehicle forward of the second air sampling point. Thus, for example, the first air sampling point may be provided near the dashboard while the second air sampling point may be provided along the headliner or in the pillars adjacent the rear window.

The air quality monitoring system may further include a third air sampling point on the motor vehicle exterior to the passenger compartment. Still further, the air quality monitoring system may include a fourth air sampling point positioned in an airstream being delivered to the passenger compartment by an HVAC system of the motor vehicle. In addition, the air quality sensor may be a particulate sensor.

Consistent with the above description, a method of monitoring and controlling air quality in a passenger compartment of the motor vehicle is provided. That method may be broadly described as comprising the step of discretely testing air quality of a first air sample taken from a first air sampling point within the passenger compartment and a second air sample taken from a second air sampling point exterior to the passenger compartment by means of a single air quality sensor. In addition, the method may include discretely testing air quality of a third air sample taken from a third air sampling point within the passenger compartment by means of the single air quality sensor. Still further, the method may include the step of discretely testing air quality of a fourth air sample taken from a fourth air sampling point positioned at an airstream being delivered to the passenger compartment by an HVAC system of the motor vehicle.

In addition, the method may include the step of directing the air stream through a filter of the HVAC system when the air quality of at least one of the first air sample, second air sample, third air sample and fourth air sample rise above a predetermined threshold level.

Still further, the method may include separately drawing the first air sample from the first air sampling point and the second air sample from the second air sampling point through the single air quality sensor by means of a single air pump. Further, the method may include providing a switching mechanism downstream from the first air sampling point and the second air sampling point and upstream from the single air quality sensor and the single air pump. In addition, the method may include controlling the switching mechanism by means of a controller. In this way it is possible to test air samples from multiple sampling points utilizing a single pump and a single air quality sensor thereby reducing system component costs, system space requirements, overall system weight and reducing system complexity.

In the following description, there are shown and described several preferred embodiments of the air quality monitoring system. As it should be realized, the air quality monitoring system is capable of other, different embodiments and its several details are capable of modification in various, obvious aspects all without departing from the air quality monitoring system as set forth and described in the following claims. Accordingly, the drawing and descriptions should be regarded as illustrative in nature and not as restrictive.

### BRIEF DESCRIPTION OF THE DRAWING FIGURE

The accompanying drawing FIGURE incorporated herein and forming a part of the specification, illustrates several aspects of the air quality monitoring system and together with the description serves to explain certain principles thereof.

FIG. 1 is a schematic block diagram illustrating the air quality monitoring system that is provided for a motor vehicle and functions to allow one to not only monitor but also control air quality in a passenger compartment of that motor vehicle.

Reference will now be made in detail to the present preferred embodiments of the air quality monitoring system, an example of which is illustrated in the accompanying drawing FIGURE.

### DETAILED DESCRIPTION

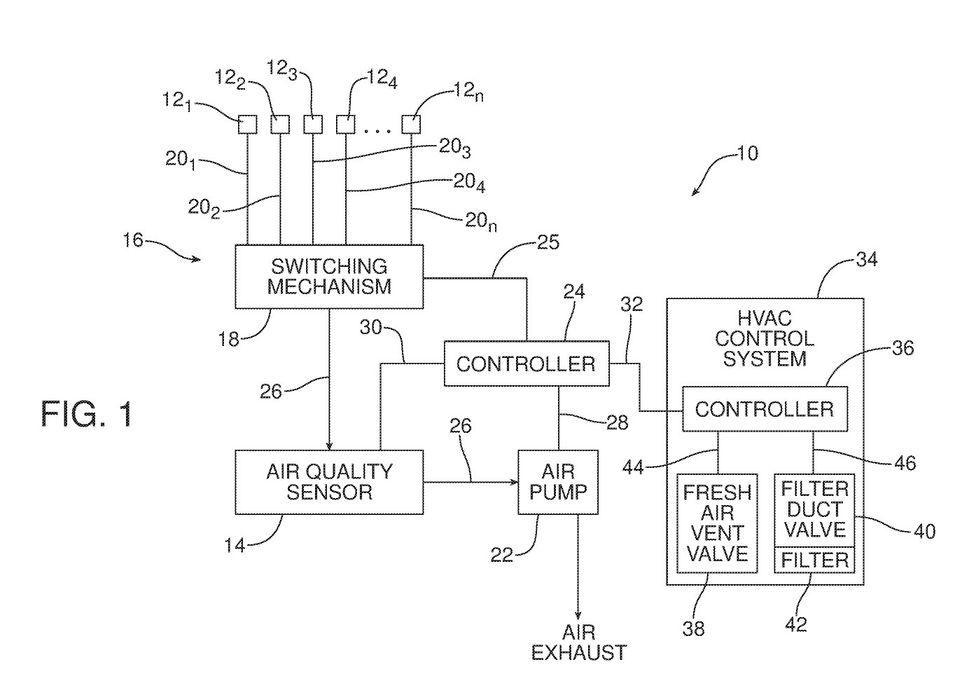

Reference is now made to FIG. 1 illustrating the air quality monitoring system 10. That air quality monitoring system 10 includes a plurality of air sampling points 12₁, 12₂, 12₃, 12₄ and 12ₙ that are provided on the motor vehicle. In addition, the air quality monitoring system includes a single air quality sensor 14 and a conduit system, generally designated by reference numeral 16, that connects the plurality of air sampling points 12₁-12ₙ with the single air quality sensor 14.

More specifically, the conduit system 16 includes a switching mechanism 18 and a plurality of hoses 20₁-20ₙ extending from the switching mechanism 18 to the plurality of air sampling points 12₁-12ₙ. One hose 20₁-20ₙ is connected to each of the air sampling points 12₁-12ₙ.

As should be appreciated, the hoses 20₁-20ₙ and the plurality of air sampling points 12₁-12ₙ are all upstream of the switching mechanism 18. In contrast, the air quality sensor 14 as well as a single air pump 22 are both located downstream from the switching mechanism 18. In the illustrated embodiment, the air pump 22 is also downstream from the single air quality sensor 14. It should be appreciated, however, that the relative position of the single air pump 22 and the single air quality sensor 14 may be reversed if desired.

As will become apparent from the following description, the air quality control system 10 allows one to take an air sample from any one of the plurality of air sampling points 12₁-12ₙ utilizing the air pump 22 and test the quality of that air sample utilizing the single air quality sensor 14. Thus, advantageously, the air quality control system 10 allows multiple point air sampling using a single air pump 22 and single air quality sensor 14. Thus, the air quality control system 10 is far less complex and expensive than air quality control systems that require the use of multiple air pumps or multiple air quality sensors in order to test air samples from multiple sampling points. The air quality control system 10 also requires less space and is lighter in weight when compared to air quality control systems incorporating multiple sensors and/or pumps.

In one particularly useful embodiment of the proposed invention, the air quality control system 10 incorporates a controller 24 in the form of a computing device such as a dedicated microprocessor or electronic control unit (ECU) operating in accordance with instructions from appropriate control software. Such a controller 24 may comprise one or more processors, one or more memories and one or more network interfaces all communicating with each other over a communication bus.

The switching mechanism 18 may comprise a plurality of electronic flow control valves and the controller 24 may be configured to control the switching mechanism including, particularly, the plurality of flow control valves, so as to allow discrete air samples to be drawn from any one of the sampling points 12₁-12ₙ through the respective hoses 20₁-20ₙ at any one time by means of the air pump 22. Further, the controller 24 may also be configured to control the operation of the air pump 22 when it is desired to take an air sample from one of the air sampling points 12₁-12ₙ.

In some embodiments of the air quality control system 10, at least one of the plurality of air sampling points 12₁-12ₙ is in a passenger compartment of the motor vehicle and at least one of the plurality of air sampling points is exterior to the passenger compartment on the motor vehicle. This allows the air quality control system 10 to take discrete samples of passenger compartment air and ambient air and test the air quality of both.

In one possible embodiment of the air quality monitoring system, the first air sampling point 12₁ is provided in the passenger compartment of the motor vehicle in the dashboard. The second air sampling point 12₂ is provided in the passenger compartment of the motor vehicle in a C pillar adjacent the rear window. The third air sampling point 12₃ is provided on the motor vehicle near the front bumper in the ambient air where it is immune to under-hood particulates. The fourth air sampling point 12₄ is positioned in a vent duct so as to be in an airstream being delivered to the passenger compartment by the HVAC system of the motor vehicle.

The controller 24 is configured to periodically draw a discrete air sample from each of the air sampling points 12₁-12₄ and test the air quality of each sample by means of the single air quality sensor 14. Thus, during the operating cycle the controller 24 could first send a control signal to the switching mechanism 18 to open an air flow path between the first hose 20₁ connected to the first air sampling point 12₁ and the air sample hose 26 extending from the switching mechanism 18 to the air quality sensor 14 and then from the air quality sensor 14 to the air pump 22. The controller 24 then sends a signal through the control line 28 to the air pump 22 so as to activate the air pump.

The air pump 22 then draws an air sample from the air sampling point 12₁ through the first hose 20₁, the switching means 18 and the air sample hose 26 through the air quality sensor 14 before exhausting that air to the environment. After a predetermined period of time required to flush the air quality control system of the air in the system at the initiation of the operating cycle, the air quality sensor 14 determines the quality of the air sample being drawn from the air sampling point 12₁ and provides data with respect to that sample to the controller 24 through the control and signal line 30.

Next, the controller 24 sends a control signal to the switching mechanism 18 through the control and signal line 25 to close the air pathway between the first air sampling point 12₁ and the air sample hose 26 and open the air pathway between the air sampling point 12₂ and the air sample hose 26. After a predetermined period of time to flush existing air from the air quality control system 10, the air quality sensor 14 tests the air quality of the second air sample being delivered from the second air sampling point 12₂ and sends a data signal respecting that quality through the control and signal line 30 to the controller 24.

Next, the controller 24 sends a control signal along the control line 25 to the switching mechanism 18 causing the closing of the air passageway between the second hose 202 and the air quality hose 26 and the opening of the air passageway between the third hose 203 and the air sample hose 26. A third air sample is then drawn from the air sampling point 12₃, through the third hose 203 and the switching mechanism 18 and the air sample hose 26 through the air quality sensor 14 and the air pump 22. After a predetermined period of time to flush the air from the second air sample from the air quality control system, the air quality sensor 14 tests the air quality of the third sample and provides relevant data to the controller 24 through the control and signal line 30.

The controller 24 then sends a control signal through the control line 25 to the switching mechanism 18 causing the switching mechanism 18 to close the passageway between the third hose 203 and the air sample hose 26 and open the air passageway between the fourth hose 204 and the air sample hose 26.

As a result, a fourth air sample is drawn from the fourth air sampling point 12₄ through the fourth hose 204 and the switching mechanism 18 and then through the air sample hose 26 through the air quality sensor 14 and the air pump 22. After flushing any remaining air of the third sample from the air quality control system 10, the air quality sensor 14 tests the air quality of the fourth sample and provides relevant data of that test quality to the controller 24 through the control and signal line 30.

At this point in time the controller 24 includes air quality data for the four discrete test samples including: (1) the first test sample from the dashboard of the passenger compartment of the motor vehicle, (2) the second test sample from the rear pillar area of the motor vehicle, (3) the third test sample from the exterior of the motor vehicle adjacent the front bumper and (4) the fourth test sample of the HVAC airstream taken from the ventilation duct.

In the illustrated embodiment, data from the four test samples is then sent from the controller 24 via the control and signal line 32 to the HVAC control system 34 of the motor vehicle and, more particularly, to the controller 36 of the HVAC control system. As is known in the art, the controller 36 of the HVAC control system 34 controls all aspects of the HVAC system of the motor vehicle. Included among those tasks and functions, the controller 36 controls the opening and closing of the fresh air vent valve 38. When the fresh air vent valve 38 is open, fresh air is drawn into the passenger cabin of the motor vehicle. In contrast, when the fresh air vent valve 38 is closed, only passenger compartment air is drawn into the HVAC system from the passenger compartment, conditioned by the HVAC system and then recycled to the passenger compartment.

In addition, the controller 36 controls a filter duct valve 40 which allows the airstream being delivered to the passenger compartment by the HVAC system to be passed through a filter 42 such as a particulate filter.

Where the air quality sensor 14 is a particulate sensor, data respecting the presence of particulates in the various air samples tested by the air quality control system 10 is shared by the controller 24 with the controller 36. Where the air particulates in any one or more of the tested samples exceeds a predetermined threshold level, the controller 36 of the HVAC control system 34 is configured to improve the air quality of the air in the passenger compartment of the motor vehicle. Thus, for example, where the third air sample of exterior air from adjacent the front bumper tests with particulate levels above the threshold level, the controller 36 will send a control signal along the control line 44 causing the fresh air vent valve to close thereby preventing the intake of additional exterior air including the particulate levels above the threshold level. At the same time, the controller 36 will send a control signal along control line 46 to the filter duct valve 40 causing air being delivered by the HVAC system to the passenger compartment to pass through the particulate filter 42 thereby filtering unwanted particulates from the airstream and creating a more healthy air environment for those in the passenger compartment.

The air quality control system 10 continuously tests air samples from the various air sampling points 12₁-12₄ and the controller 36 adjusts the fresh air vent valve and the filter duct valve 38, 40 accordingly. Thus, for example, when the particulate levels of the exterior air as indicated by sample 3 return to acceptable levels, the controller 36 may again open the fresh air vent valve 38 to allow fresh air to mix with passenger compartment air. Further, the controller 36 may adjust the filter duct valve 40 so that the HVAC airstream bypasses the filter 42 when particulate filtration is not required.

The foregoing description should be considered as illustrative in nature and not limiting in scope. As should be appreciated, substantially any number of air sampling points 12₁-12ₙ may be provided. These may be located at various places in the interior and exterior of the vehicle. For example, in the vehicle interior, an air sampling point 12₁-12ₙ may be provided on the instrument panel, including behind the Class A surface such as at the HVAC recirculation inlet (which has a potentially higher movement of air within the passenger compartment space), in styled grilles or hidden openings on the Class A surface, at the center stack, at the driver steering wheel column area, at the HVAC case outlets, such as panel, floor, defrost, lap cooler, demister, other bleeds (to potentially sense and indicate the filtered/cleaned status of air exiting the HVAC case) and on the passenger side by the glove box. An air sampling point 12₁-12ₙ may be provided on the headliner or in any generally overhead position or integrated within the overhead console, storage bins, lighting assemblies or HVAC controls. An air sampling point 12₁-12ₙ may be provided on the center storage console to allow easy sensing of the second row cabin space, next to a bed in a sleeper cab of a heavy truck for reading particulate levels for several hours while an operator is sleeping, near a cooking stove in an RV camper (where an operator chooses for the vehicle to have an HVAC system that automatically activates an air cleaning hood or exhaust fan when a predetermined particulate level is exceeded), or on trim pieces.

If placed on trim pieces at both front and rear spaces in a passenger compartment, a comparison can be made for air particulate levels at the front and rear of the passenger compartment to determine if an increase is due to addition of particulates from an open front or rear window or due to the presence of a smoker in the motor vehicle. Such information garnered by the air quality control system 10 may be indicated to the operator of the motor vehicle through a voice processor or human interface display screen such as commonly associated with a body control module of a motor vehicle.

An air sampling point 12₁-12ₙ may be provided on the upper B pillar where it is close to the motor vehicle operator's nose and would, in turn provide a more accurate indication of the particulate levels of the air near the ingestion point of the motor vehicle operator. This would provide for faster and more accurate reaction and in turn result in higher confidence in the operation of the air quality control system.

Further, it should be appreciated that an air sampling point 12₁-12ₙ may be placed on the vehicle exterior at various locations including, for example, near the front bumper where such a sensor would be immune from under-hood particulates, in an air intake where the sensor would monitor air being taken into the HVAC system and that information may be used to improve filter life by reducing usage of fresh mode when high particulates are detected.

An air sampling point 12₁-12ₙ may be provided near the top of the roof of the motor vehicle where it would be protected from high particulate readings commonly expelled from exhausts pipes of motor vehicles adjacent the ground. An air sampling point 12₁-12ₙ may be provided under the side mirrors for best proximity to open windows or under hood for monitoring air particulates produced by the motor vehicle.

As noted above, the air quality sensor 14 may be a particulate sensor. Alternatively, it may be a carbon dioxide sensor or substantially any other sensor providing information of interest and useful to the operator of the motor vehicle. The terminology “single air quality sensor” as used in this document may refer to a single sensor assembly incorporating multiple sensors such as a carbon dioxide sensor and a particulate sensor. The significance of the term “single” relates to the fact that a plurality of air sampling points 12₁-12ₙ are monitored and tested by a single air quality sensor or sensor array just as a single air pump 22 is utilized to draw air from those plurality of air sampling points while the switching mechanism 18 allows for discrete testing of each air sample from each air sampling point.

Advantageously, the air quality control system 10 provides a number of benefits and advantages. By utilizing a single air quality sensor or sensor array 14 and a single air pump 22 to discretely test air samples from a plurality of air sampling points 12₁-12ₙ, system costs are reduced, system weight is reduced, energy consumption is reduced, noise and vibration are reduced and overall reliability is improved.

As should be appreciated, each hose 20₁-20ₙ has a distal end at one of the plurality of air sampling points 12₁-12ₙ and a proximal end at the switching mechanism 18. Each hose 20₁-20ₙ is flexible and relatively small in size allowing air sampling points 12₁-12ₙ to be placed at locations that would not accommodate an air quality sensor. This increases the overall availability of potential sensor locations. Further, it should be appreciated that by using a single air quality sensor or sensor array 14, the single air quality sensor or sensor array may be placed at a protected location and only the ends of the hoses 20₁-20ₙ are exposed to environmental conditions at the air sampling points 12₁-12ₙ. Obviously, the hoses 20₁-20ₙ may also be easily passed through apertures in sheet metal and bulkheads in order to reach desired air sampling points 12₁-12ₙ.

The foregoing has been presented for purposes of illustration and description. It is not intended to be exhaustive or to limit the embodiments to the precise form disclosed. Obvious modifications and variations are possible in light of the above teachings. All such modifications and variations are within the scope of the appended claims when interpreted in accordance with the breadth to which they are fairly, legally and equitably entitled.

What is claimed:

1. An air quality monitoring system for a motor vehicle, comprising:
a plurality of air sampling points on said motor vehicle;
a single air quality sensor; and
a conduit system connecting said plurality of air sampling points with said single air quality sensor.

2. The air quality monitoring system of claim 1, wherein said conduit system includes a switching mechanism and a plurality of hoses extending from said switching mechanism to said plurality of air sampling points.

3. The air quality monitoring system of claim 2, further including an air pump.

4. The air quality monitoring system of claim 3, wherein said single air quality sensor and said air pump are downstream from said switching mechanism.

5. The air quality monitoring system of claim 4, wherein an air sample hose connects said switching mechanism with said single air quality sensor and said air pump whereby a discrete air sample from any one air sampling point of said plurality of air sampling points is tested for air quality by said single air quality sensor.

6. The air quality monitoring system of claim 5, further including a controller configured to control said switching mechanism and allow said discrete air sample to be drawn from said any one air sampling point by said air pump.

7. The air quality monitoring system of claim 6, wherein said controller is further configured to control said air pump.

8. The air quality monitoring system of claim 7, wherein a first air sampling point of said plurality of air sampling points is in a passenger compartment of said motor vehicle and a second air sampling point of said plurality of air sampling points is exterior to said passenger compartment.

9. The air quality monitoring system of claim 7, including a first air sampling point in a passenger compartment of said motor vehicle.

10. The air quality monitoring system of claim 9, including a second air sampling point in said passenger compartment of said motor vehicle wherein said first air sampling point is motor vehicle forward of said second air sampling point.

11. The air quality monitoring system of claim 10, including a third air sampling point on said motor vehicle exterior to said passenger compartment.

12. The air quality monitoring system of claim 11, including a fourth air sampling point positioned in an air stream being delivered to said passenger compartment by an HVAC system of said motor vehicle.

13. The air quality monitoring system of claim 12, wherein said single air quality sensor is a particulate sensor.

14. A method of monitoring and controlling air quality in a passenger compartment of a motor vehicle, comprising:
discretely testing air quality of (a) a first air sample taken from a first air sampling point within said passenger compartment and (b) a second air sample taken from a second sampling point exterior to said passenger compartment by means of a single air quality sensor.

15. The method of claim 14, including discretely testing air quality of a third air sample taken from a third air sampling point within said passenger compartment by means of said single air quality sensor.

16. The method of claim 15, including discretely testing air quality of a fourth air sample taken from a fourth air sampling point positioned in an airstream being delivered to said passenger compartment by an HVAC system of said motor vehicle.

17. The method of claim 16, including directing said airstream through a filter of said HVAC system when air quality of at least one of said first air sample, said second air sample, said third air sample and said fourth air sample rise above a predetermined threshold level.

18. The method of claim 14, including separately drawing said first air sample from said first air sampling point and said second air sample from said second air sampling point through said single air quality sensor by means of a single air pump.

19. The method of claim 18, including providing a switching mechanism downstream from said first air sampling point and said second air sampling point and upstream from said single air quality sensor and said single air pump.

20. The method of claim 19, including controlling said switching mechanism by means of a controller.
